(19) United States
(12) Patent Application Publication
(10) Pub. No.: US 2002/0165515 A1
(43) Pub. Date: Nov. 7, 2002

(54) Multi-use diaper

(76) Inventor: Herbert R. Burnham, Chula Vista, CA (US)

Correspondence Address: Herbert R. Burnham, 2252 Boulder Creek Street, Chula Vista, CA, 91915, US

(21) Appl. No.: 09/846,229

(22) Filed: May 2, 2001

(51) Int. Cl. A61F 13/15; A61F 13/20

(52) U.S. Cl. 604/385.14; 604/385.13

(57) ABSTRACT

The invention is a diaper comprising an impermeable outer shell, means for securing it around the waist and legs, and a set of multiple absorbent pads located inside the shell. Each such pad in the set being positioned and adapted to receive and hold liquid and solid waste in turn. Each pad being easily removable after use, through suitable openings in the shell, leaving behind a clean pad in its place for subsequent use and removal. Each said pad comprising a top layer of absorbent material lined with a lower, thin layer of impermeable material designed to hold liquids and keep them from seeping downward to the next pad in the stack, an end tongue or handle for puling out of the pad stack and out of the shell, and an integral (plastic) bag or enclosure to isolate the used pad. ?

### BACKGROUND AND FIELD OF THE INVENTION

Disposable diapers are widely used by infants, incontinent adults and by patients in hospitals. Said diapers have improved significantly in the last twenty years or so in order to incorporate desirable characteristics such as enhanced liquid retention in the absorption area and a watertight closure about the legs and waist. Other characteristics have been added to the essential design to make disposable diapers ever more practical. For example, visible indicators telling when the diaper is wet and must be changed, better or different linings that make them more comfortable, aromatic aids to make the surroundings more pleasant; and many more additions still.

The basic design of the disposable diaper, however, has not changed; essentially, it remains disposable after one use. One-use disposable diapers are now handy and difficult to do without. At the same time, they are also costly and must be dumped, adding to problems related to landfills in particular and to those of the environment in general.

It is noteworthy that in spite of all improvements, all diapers are not entirely practical in all circumstances. For example, changing a diaper in a crowded area such as a shopping mall is difficult since it involves undressing the user in order to put on a new diaper. Also, carrying replacement diapers may be inconvenient, just as disposing of the used diaper may not be easy in some cases because of its bulk and smell. Undressing a baby in other circumstances such as inside a small car, in an airplane lavatory or in cold weather, can be awkward. Adult users may find it difficult as well in similar and in different circumstances. Hospital nurses must dedicate a great amount of effort and time just to keep conventional diapers timely disposed and refreshed.

The invention solves these and other problems while reducing waste and related expenses.

### OBJECTS OF THE INVENTION

The object of the invention is to provide a multiple use diaper.

Another object is an easy change, easy clean diaper. Yet another object is reducing cost and waste in the use of disposable diapers.

Still another object is the incorporation of the multi-pad design, as described herein, into the fields of feminine hygienic pads and bladder control.

### IN THE DRAWINGS

FIG. 1 is a perspective view of the invention showing an open diaper with its internal pad stack and front openings for retrieving used pads.

FIG. 2 is a cut-away side view of a single pad.

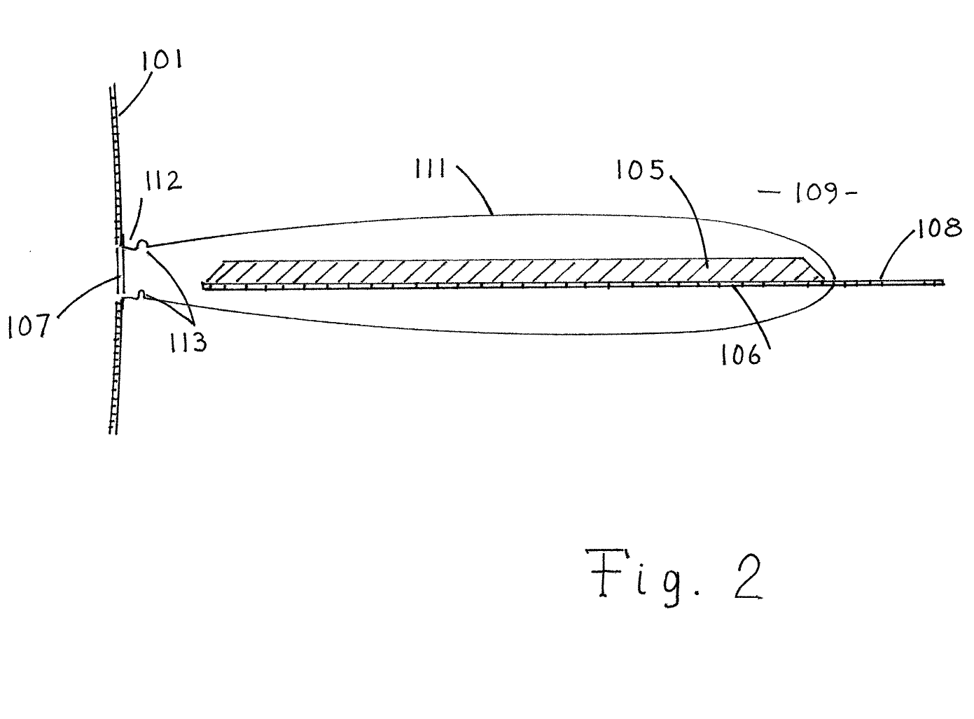

### DETAILED DESCRIPTION OF THE INVENTION

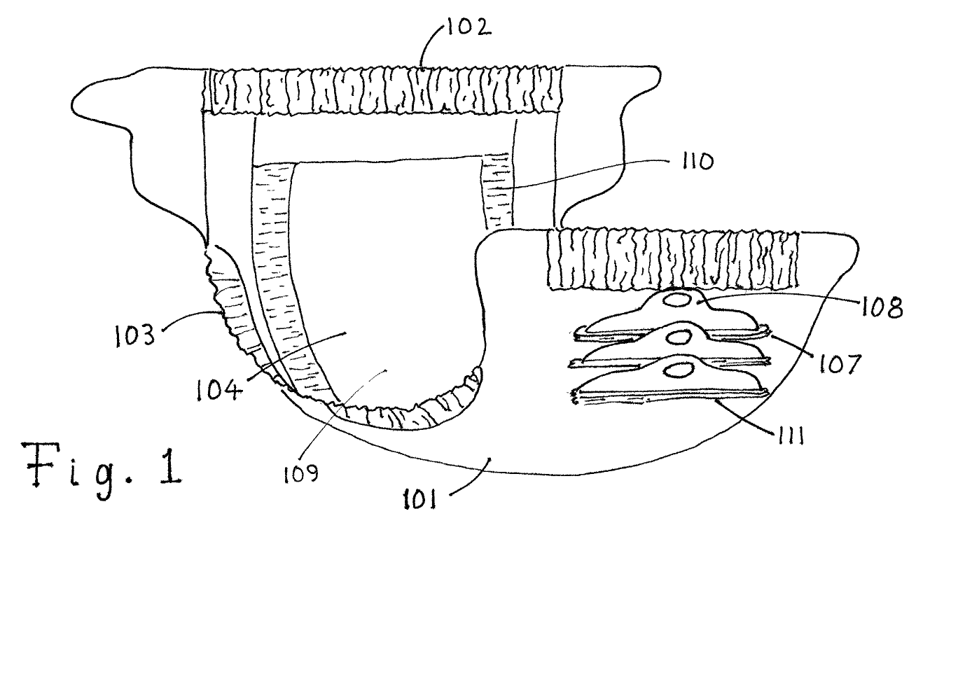

The invention, as shown in FIG. 1, is a multi-use diaper consisting of an impermeable outer shell 101 shaped to close around the waist 102 and around the legs 103 of the user. Located inside shell 101 is a stack or pile of pads 104.

Pad stack 104 is a set of disposable pads designed to receive human liquid and solid waste and is located within the enclosure defined by shell 101. Each set of pads 104 consists of a multiplicity of absorbent pads. Each said pad comprises absorbent portion 105, impermeable lining 106, tongue 108 and integral bag 111. Said bag 111 is used to totally enclose and isolate a used pad before disposal.

Whenever a pad becomes wet or soiled, it can be removed leaving in place a new and unused pad ready to receive liquid and solid waste.

In one embodiment of the invention, the outer shell 101 must be opened to remove the used pad.

Shell 101 itself may be lined inside with different materials for comfort and fitting; these characteristics are not illustrated in the drawings.

In another embodiment of the invention, shell 101 comprises one or more openings 107 designed to facilitate the withdrawal of a used pad 109 therethrough. When using this opening, it is not necessary to open up the entire diaper at waist 102, since used pad 109 can be removed by grasping tongue 108 and pulling it out through opening 107. This will leave the next pad in stack 104 in place ready for use.

Pad 109 in stack 104 may be held in place with tiny breakaway ties, not shown in drawings, which snap and release their corresponding pad as it pulls away. Elastic guides or folds 110, shown in FIG. 1 help keep stack 104 in place and provide a comfortable fit.

Tongue 108 may bear a suitable indication such as a number, a letter or an illustration to advise quantity of pads 109 already spent and quantity thereof remaining in stack 104.

Two openings 107 may be provided, in front and back of shell 101, to improve access to and retrieval of a used pad 109. In this embodiment, pad 109 comprises two tongues 108, one at each end.

Set of pads 104 may consist of a simple multiplicity of pads, in one essential embodiment of the invention. Other combinations are possible, such as the incorporation of cleansing wipes, not shown but resembling pad 109 in shape and size, interposed between pads 109 to wipe and clean as they are removed, assuring a fresh start for the new pad 109. Similarly, said cleansing wipes may consist of one or more portions to provide a sequence of cleansing and deodorizing functions as they are removed.

In another embodiment of the invention, an integral plastic bag 111 is provided with each pad to enclose or wrap around said pad as it is being pulled out through opening 107. Thus, each pad 109 incorporates bag 111, which is a tubular, or elongated bag suitably wrapped around and fastened to one end of pad 109 at the point where absorbent portion 105 ends and tongue 108 begins. Said bag 111 is secured, at its other end, to shell 101. When tongue 108 is grasped and pulled to retrieve used pad 109, said pad is enveloped by bag 111 as said pad is being pulled out. Thus, when pad 109 is totally outside shell 101, it is totally inside bag 111. At this point, bag 111 is separated from shell 101 at breakaway area 112. Additionally, bag 111 may comprise integral closure means 113 to seal in pad 109. Alternately, bag 111 may be designed without attachment to shell 101, to be pulled manually around pad 109 by hand.

The invention as described above may be used in the field of disposable diapers in one practical embodiment. It is not limited to this application, however, since it may be incorporated to a more permanent garment. Such a permanent garment would use more convenient and/or expensive and/or specific materials adapted to different climates or activities. Thus, a re-usable leather diaper which may be found more adequate for some uses, or a diaper shell padded for comfort or cold weather, are logical incorporations of the invention.

Likewise, a pad stack alone, as described herein, may be used in the field of feminine pads and/or in combination with feminine underwear. Similarly, it will be useful in the field of light bladder control. The combination of feminine underwear, permanent or disposable, and a pad stack as described herein, is an ideal incorporation of the invention. Still more, this invention may be profitably incorporated in diverse underwear used in hospitals, clinics and wards where a quick change without need of undressing a patient will surely be appreciated.

1. A multi-use diaper consisting of an outer impermeable shell shaped to fit about a user, means to secure it in place and having internal absorbent means to receive and hold human liquid and solid waste, wherein the absorbent means is a set of multiple, disposable, absorbent pads, each individual pad designed to receive and hold liquid and solid waste, and each said individual pad being removable from its set through suitable openings in the impermeable shell, leaving a next disposable pad in the set in similar position to receive liquid and solid waste and be removed and disposed in turn.

2. The diaper of claim 1 wherein each pad comprises an upper absorbent portion lined with a lower impermeable material, one or more tongues for grasping and removing and an integral impermeable bag for disposal.

3. The diaper of claim 2 wherein said disposal bag is affixed in a tear away relation to the diaper shell at one end, and at the other it is suitably affixed and wrapped around the pad at the juncture where the tongue for grasping and the absorbent layer meet, said bag further comprising a closure means to seal in and isolate a used pad.

4. A garment such as a panty wherein a pad stack comprising a multiplicity of disposable, liquid absorbing pads is incorporated. ? ?
